(19) United States
(12) Patent Application Publication
(10) Pub. No.: US 2016/0161149 A1
(43) Pub. Date: Jun. 9, 2016

(54) CHEMICALLY BASED HEATER FOR A BIO-MECHANICAL DEVICE AND ARTICLE TO BE HEATED

(71) Applicant: RECHARGEABLE BATTERY CORPORATION, College Station, TX (US)

(72) Inventors: Adam Laubach, Kingwood, TX (US); Christopher S. Pedicini, Nashville, TN (US); John Beckerdite, College Station, TX (US); Darko Marquez, College Station, TX (US)

(21) Appl. No.: 14/559,447

(22) Filed: Dec. 3, 2014

(51) Int. Cl. F24J 1/00; A61F 5/058; A43B 7/02; H01M 6/36; A41D 13/005

(52) U.S. Cl. CPC F24J 1/00; H01M 6/36; A41D 13/0051; A43B 7/02; A61F 5/058

(57) ABSTRACT

An article capable of being heated having a material with a plurality of cavities formed therein. The article includes at least one heater which may be embedded in at least one of the plurality of cavities formed in the material. A sheet of porous material may be operatively engaged with the article over the material so that the plurality of cavities are covered and the at least one heater is held therein.

### RELATED APPLICATIONS

None.

### TECHNICAL FIELD

The present invention generally relates to heaters and articles having heaters embedded therein. More specifically, the present invention relates to substantially dry oxygen activated heaters and thermoformable articles having heaters embedded or engrained therein.

### BACKGROUND OF THE INVENTION

Thermoformable articles typically become moldable or formable when a heat source is applied to them. In some known articles, a heater or the like may be included with or coupled to the article. For example, in pending but unpublished applications to Rechargeable Battery Corporation, a thermoformable article in the form of a splint is disclosed wherein the article includes a heater which couples to the exterior of the article. While attaching the heater to the exterior of the article provides satisfactory results, particularly for thinner or smaller articles, the heating of the article resulting from the surface heater may not be uniform and may not substantially penetrate the article quickly. This may result in longer heating times, articles which do not uniformly heat for molding, and may also result in higher molding temperatures, which in turn may lead to longer setting times or damage to the article. Additionally, placing the heater on just the outer surface of the article means that only a portion of the heat generated by the heater is transmitted to only a portion of the article. For thicker articles, the heat may not successfully transfer heat throughout the article, causing at least a portion of the article to remain static while the rest is moldable.

The heaters used for thermoformable articles are typically oxygen activated heaters like those described in the pending but unpublished applications to Rechargeable Battery Corporation. In addition to using the oxygen activated heaters for thermoformable objects, they may have numerous other uses, like, for example, heating food and as hand, foot or body warmers. Known oxygen activated heaters are typically manufactured using a wet process with a material which includes zinc, carbon, an optional binder, and water. The heater mix is rolled into sheets and dried in an oven. As the water evaporates from the sheets in the oven, voids are created within the sheets. During the subsequent activation of the heater, resulting in a reaction between the zinc and oxygen, the voids provide the porosity required to contact the zinc in an efficient manner.

The wet process has at least one key advantage in that it yields a heater sheet with sufficient structural integrity that it can be handled, placed, and utilized in a variety of ways. However, while creating a satisfactory heater, the wet process is time consuming in both preparation and drying, with the drying being the primary bottleneck in the production process. The additional time lowers production throughput and increases costs.

The alternative to the wet process is a dry process in which water is not, or is substantially not, used to produce the heater. However, there has not been a dry process for manufacturing a heater developed which can match the mechanical integrity and performance of a wet process heater.

Therefore, it would be advantageous if an oxygen activated or chemical heater could be manufactured using a dry process that substantially or completely removes water and any necessity to dry the heater once constructed.

It would also be advantageous to create a thermoformable article which is integrated with a heater in a manner which allows for more efficient exchange of heat between the heater and the article, and which insures that the entire article is heated uniformly, as quickly as possible.

The present invention is provided to solve these and other issues.

### SUMMARY OF THE INVENTION

The present invention is directed to a heater and an article having a heater embedded within the article. The heater may be an oxygen activated or chemical heater which is manufactured using a dry process, resulting in a heater which has a performance which matches that of a heater manufactured using a wet process.

According to one aspect of the invention, an article capable of being heated is provided. The article is formed using a material which includes at least one cavity formed therein. The article further includes at least one heater which is embedded in at least one of the plurality of cavities formed in the material. The article finally has a sheet of porous material operatively engaged with the material so that the at least one cavity is covered by the sheet of porous material and the heater material is held therein. In order to maximize surface area and provide the necessary cavities, the material may be formed in a specific configuration or manner, like for example, a honeycomb configuration.

The material forming the article may be a non-thermoformable material or a thermoformable material with any heat generated by the heater making the thermoformable material moldable. The material may also be a woven or non-woven material.

The heater included with the article may also be a heater mix forming a chemical or oxygen activated heater. At least a portion of the heater mix may be embedded in one, a portion, or all of the plurality of cavities. These cavities serve to confine the heater mix and during preparation and use. For example, if the article is cut or trimmed subsequent to activation the amount of heater mix available to be released is minimized. The cavities also provide additional surface area for the transfer of the heat to the article. The heater mix may further include an electrolyte being included therein in order to trigger activation. The article may be sealed within an airtight container in order to prevent oxygen or some other chemical from contacting the heater, and may include an insulator material which substantially surrounds and insulates the thermoformable material when thermoformable material is used.

The heater may also or alternatively be activated using one or more methods. For example, the heater may be activated using microwaves or an induction process.

When constructed as a chemical or oxygen activated heater using a dry process, the heater mix may include zinc, carbon, and binder, as well as some amount of water less than 2% of the total weight of the heater. The heater mix may also include an electrolyte, a binder, and/or a filler. The heater mix preferably has a density in the range of 0.5 g/cm³ and 1.8 g/cm³.

According to another aspect of the invention, the article may be trimmable or scalable while retaining a substantial portion of any heater embedded in the article.

Other advantages and aspects of the present invention will become apparent upon reading the following description of the drawings and detailed description of the invention.

### BRIEF DESCRIPTION OF THE DRAWINGS

FIG. 1 shows a perspective view a portion of an article to be heated as contemplated by the present invention;

FIG. 2 shows an exploded view of a portion of an article to be heated as contemplated by the present invention;

FIG. 3 shows a perspective view of an article to be heated sealed inside a container; and

FIG. 4 shows a graphical representation of the heating and cooling of an article as contemplated by the invention.

### DETAILED DESCRIPTION OF PREFERRED EMBODIMENTS

While this invention is susceptible to embodiments in many different forms, there is described in detail herein, preferred embodiments of the invention with the understanding that the present disclosures are to be considered as exemplifications of the principles of the invention and are not intended to limit the broad aspects of the invention to the embodiments illustrated.

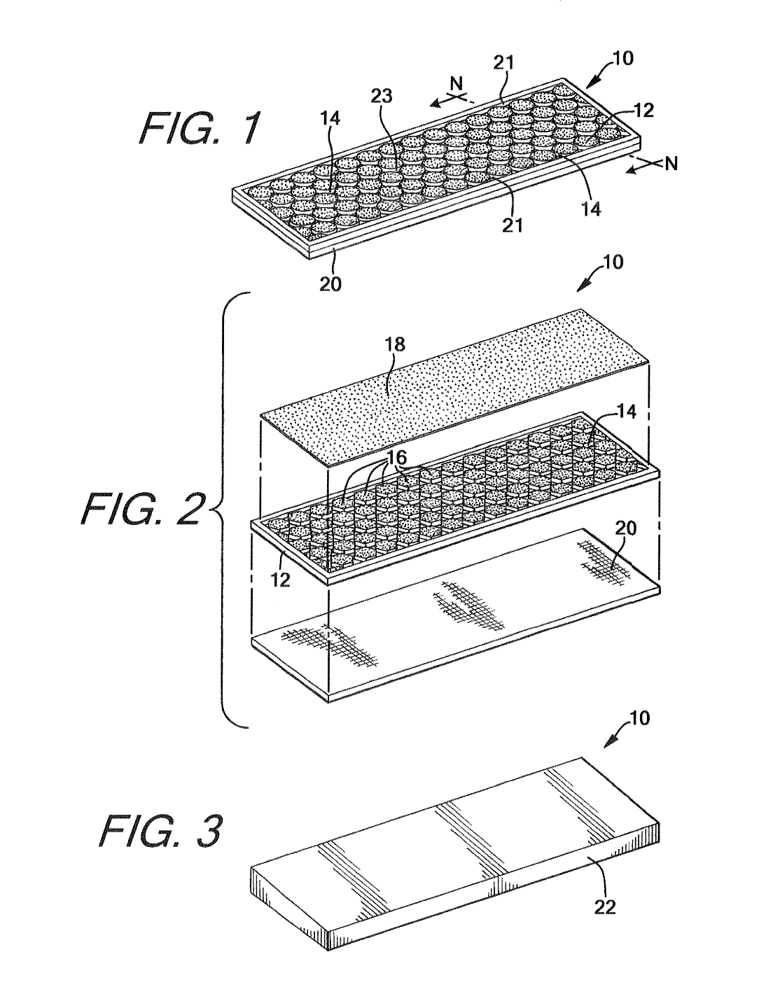

FIGS. 1 and 2 show an exemplary embodiment of an article capable of being heated as contemplated by the invention. Article 10 includes material 12 forming the article. Material 12 includes at least one cavity 14 formed therein. A plurality of cavities 14 are shown in FIGS. 1 and 2 as being both a network of circular openings (see FIG. 1) and in a honeycomb configuration (see FIG. 2). However it should be understood that when a plurality of cavities are provided, the cavities may be configured in any formation, size, or configuration and accomplish the purposes of the present invention. In one simple form, there may be only one cavity resulting in a tray which holds the heater contents. Additionally, the distribution of cavities need not be uniform. For example, where it is desired to provide more heat to the outer edges of the article the cavities may be larger to allow for the addition of more heater material. The cavities may also be completely or substantially confined to one side of the article of only one side is to be heated.

Article 10 further includes at least one heater 16 (shown in FIG. 2 as a heater mix) which is embedded within at least one cavity 14 of material 12. Though shown in only a few cavities, at least one or a portion of the heater may be embedded in fewer or more cavities as is required for the particular article or use. For many applications, in order to insure the fastest, most efficient heat transfer, at least one heater—or as will be discussed herein a portion of heater mix—may be embedded in each cavity so the entire article is uniformly heated substantially simultaneously. Of course the amount of heater embedded in each particular cavity or a region of cavities may vary. For example, the amount of heater embedded in cavities located in a perimeter portion or edge, like for example perimeter 21, may be greater than the amount of heater embedded in cavities located in a middle portion or area, like for example middle 23. Other configurations or amounts of embedded heater may be realized by the invention, like for example a different particular area, side, or cavity having more or less heater embedded therein.

In order to hold in heater(s) 16 or any portion of heater mix, article 10 may further include at least one sheet of porous material 18 which may substantially cover and be placed over material 12, over top of cavities 14. The sheet of porous material may be constructed to allow one or both of oxygen and electrolyte fluid to pass through the sheet and reach the article or heater as required. In addition to allowing oxygen and electrolyte through, the porous sheet of material may allow for the article to be trimmed along axis n (the narrow axis of the article, for example) while the at least one cavity or the plurality of cavities retain a substantial amount of the heater located in the portion of the article to be used. Retaining a substantially portion of the heater, like for example 95% or more, will allow for the trimmed article to generate any required or desired amount of heat for the remaining portion of the article. The scalability will allow for articles to be designed and then sized on site or when needed, rather than having to create custom sized articles. When paired with thermoformable material as will discussed further herein, a single article like a splint may be provided which can be sized and formed to match a substantial number of individuals rather than the need to buy a custom sized splint or carry multiple splints having different sizes.

As also shown in FIGS. 1 and 2, article 10 may also include insulator material 20 which surrounds and covers at least a portion of article 10 and material 12. The insulator material may be adhered to a single side or portion of article 10, like for example the bottom or a portion which may contact a user's skin or some other material which acts as an intermediary. Alternatively, the insulator may substantially surround all but the porous material, or even surround, or in some cases be formed as, the porous material. The insulator material may be, for example, a felt or other fabric material which may be at least partially flexible while providing a heat barrier between the article and contact surface of a body to which the article is attached.

The material utilized in the article may be woven and/or non-woven material, and may be either thermoformable or non-thermoformable material, depending on the particular article. In one embodiment of the present invention, material 12 may be a thermoformable material which may become moldable or malleable in response to heat generated by heater(s) 16 once the heater(s) are activated within article 10. Thermoformable material which may be used as the material forming a portion of the article may include, for example, poly(lactic acid), ethylene vinyl acetate, poly(caprolactone), poly(hydroxybutyrate), polyethylene, polypropylene, polystyrene, and any combinations thereof. It is contemplated that the thermoformable material may optionally contain fillers to minimize cost and/or improve mechanical properties such as modulus. A filler that can improve the thermal conductivity of the material may also be employed. Where thermoformable material is used for the material, the thermoformable material and heater(s) should be configured such that the thermoformable material is heated to a moldable state by the heater in approximately 90 seconds. Once heated, the temperature of the article and heater should drop below the temperature required to mold the thermoformable material in less than 10 minutes from the time of activation. Within ten minutes of the activation of the heater, the heat produced by the heater and retained within the article should be lowered to a point where the material is no longer moldable and is in a substantially static, molded position wherein the article has returned to a desired level or rigidity.

Though a portion of a heated article is shown in FIGS. 1 and 2, it is contemplated by the invention that the material used to form a portion of the article—whether thermoformable or otherwise—may be configured in any desired manner. For example, material 12 may be formed into a splint, a sleeve, a sock, a glove, a shirt or a portion of a shirt, a vest, a pant, portion of a pant or a single legging, a hat or other headwear or portion of a hat or other headwear, armor or a portion of an armor to be worn by an individual, a brace or portion of a brace, a prosthetic or portion of a prosthetic, or an exoskeleton device.

Depending on the use of the article and the material and desired properties, the embedded heater(s) may have different characteristics and forms. For example, if the primary purpose of the heater and article is to provide heat to a body, the heater(s) embedded in one or more cavities within the article may be configured and designed to remain activated for a longer period of time. However, if the material used in the article is thermoformable and the article is intended as a thermoformable article, or if the heater is only intended to provide heat for a short amount of time, the heater may quickly increase and decrease heat production. The quick increase and decrease in temperature may allow any thermoformable material to reach the moldable temperature and allow the material to cool in a substantially formed position and state quickly.

Whether the material is thermoformable or not, heater 16 may be constructed as a heater mix or a chemical mix which may react when introduced to additional chemicals or stimuli to generate heat. For example, heater 16 may be constructed as a heater mix which acts as an oxygen activated heater which is manufactured using a substantially dry process. Where an oxygen activated heater is used in article 10, an exterior container or pouch 22 (shown in FIG. 3) which is airtight or impermeable by air may be included in order to prevent unwanted or premature activation of the heater. Though an oxygen activated heater is preferred, it is contemplated that heater 16 may be activated in other manners, like for example using microwaves or induction heating.

In order to manufacture an oxygen activated heater or heater mix using a dry process, a combination of chemicals or compounds may be mixed and combined. The heater mix may be, for example a mixture having approximately 70.0%-90.0%, and more specifically approximately 80.0%, zinc or other chemical which is reactive to oxygen, approximately 5.0%-15.0% carbon, approximately 0.0%-20.0% polytetrafluoroethylene (“PTFE”) acting as an optional binder, and approximately 0.0%-5.0% water with no more than 5% water. A filler may be optionally added to the mix to increase the density of the heater mix. The combination of zinc or other oxygen reactive chemicals, carbon, any optional binder, any optional filler, and any water should be within the bulk density range of 0.5-2.0 g/cm³.

While zinc is the preferred active chemical within the heater because of its ability to quickly provide high amounts of heat once exposed to oxygen and activated, it is contemplated that other chemicals may be utilized, including but not limited to, aluminum, copper, or iron. For the optional binder, rather than, or in addition to, PTFE, polyethylene may be used. When an optional filler is included to alter the density of the heater mix, the optional filler may be sawdust, wood pulp, paper products, cotton linters, ground seed or nut hulls or products, expanded perlite, vermiculite, diatomaceous earth, open-cell polyurethane foam, poly(acrylic acid), hollow beads or spheres, or some combination thereof.

Each of the chemicals or compounds may be provided into a mixer, like for example a rotary mixer, and mixed for a period of time in order to combine each component. Since the mixture is constructed using a completely or a substantially dry process, there is no need to dry the mixture in an oven.

In order for the heater to activate once oxygen is introduced, the heater mix may include an electrolyte which may be added to the mix once the chemicals and compounds are combined. For heaters which are not manufactured with an electrolyte, an electrolyte may be added after the heater is placed, like for example in a cavity of an article as discussed herein. The electrolyte may be added directly to the heater(s), or may be added through a porous member or the like after the porous member is fit over the heater and any article which includes a heater. Whether included in the heater mix or later added, electrolytes which may be used include, but are not limited to, sodium chloride, sodium bromide, potassium chloride, potassium bromide or potassium hydroxide. The preferred amount of electrolyte to be added to the heater mix ranges from 15%-40% by weight of the heater mix. For example, for 10 g of heater mix, it is preferable to have 30% loading, which requires approximately 3 g of electrolyte. The concentration of the electrolyte used with the heater mix should range from 1%-40% by weight of solution.

As with any heater or heater mix, the amount of electrolyte added to each particular cavity or a region of cavities may vary. Following the example above, the amount of electrolyte added to heater embedded in cavities located in a perimeter portion or edge, like for example perimeter 21, may be greater than the amount of electrolyte added to heater embedded in cavities located in a middle portion or area, like for example middle 23. The amount of electrolyte added to each cavity or region of cavities may be selected based upon the amount of heater in each cavity, or to incur a desired result or heating time for a particular cavity or region in the article. Rather than add more or less electrolyte to a particular cavity or region, the concentration of the electrolyte added to any particular cavity or region of cavities may be varied. Electrolyte having a higher concentration, like for example 40% by weight of solution, may be added to cavities along perimeter 21, while electrolyte having a concentration of 20% by weight of solution may be added to cavities located in middle 23. Once the heater mix is combined and any electrolyte is added, if the heater mix is to be utilized as a standalone heater without an article, the heater may be packaged in an air impermeable or airtight container. The container may include internal dividers or compartments to prevent the movement of the heater mix, and may be constructed from polylactic acid), ethylene vinyl acetate, poly(caprolactone), poly(hydroxybutyrate), polyethylene, polypropylene, polystyrene, or combinations thereof. At least one portion of any package housing a standalone heater should have a material with a thermal conductivity of at least 10 W/mK in order to provide for satisfactory heat transfer once the heater is activated. The package should also provide for a tear away or other access point to allow the heater mix to be exposed to oxygen in order to activate the heater. The standalone heater packaging may also include at least one attachment element which will allow the heater to attach to a body or device. The attachment element may be a physical element like a clip or pin which permits the heater to be attached directly to a body or device, or may be an adhesive or other coating which allows the heater to be coupled to a body or device. The packaging may also be flexible in order to allow for the manipulation of the heater to achieve a particular shape or configuration.

If the heater mix is instead intended for use in an article like those discussed herein, the article may then be constructed as follows. It should once again be understood that though a thermoformable splint will be used as an example, non-thermoformable materials, and articles other than a splint may be constructed in a similar manner. First the material may be designed and configured, like for example a thermoformable material formed as a splint with a honeycomb surface. Next, the heater mix may be used to cover and fill the honeycomb surface or configuration, placing the heater mix into each of the cavities. Excess heater mix may be removed so that the heater mix is uniformly formed within the cavities. The sheet of porous material may then be placed over the honeycomb surface and adhered thereto in order to lock in the heater mix and prevent it from escaping. If the heater mix does not include any electrolyte, electrolyte may then be added to the heater mix through the sheet of porous material. The article may then have any insulation attached to the article, and then be packaged in an air impermeable or airtight container until ready for use.

In operation, once the article is removed from the air impermeable or airtight packaging, the combination of the heater mix and electrolyte will cause the heater to begin heating. If the material which forms part of the article is not a thermoformable material, the heat will be transmitted to and through the article. If the material forming part of the article is a thermoformable material, the thermoformable material should be provided with enough heat to make the material moldable in a very short period of time, preferably 180 seconds or less, more preferably 90 seconds or less. For example, if the article is a splint, the splint will preferably become moldable in about 90 seconds so that it can quickly be formed to a body. The heat generated by the heater should quickly dissipate and the heater should then cool off and put the article in a substantially static, formed shaped, as desired. Since the heater is embedded within the article, the heat transfer will be quicker, more efficient, and more uniform, throughout the material and article, allowing for better and quicker molding of the article.

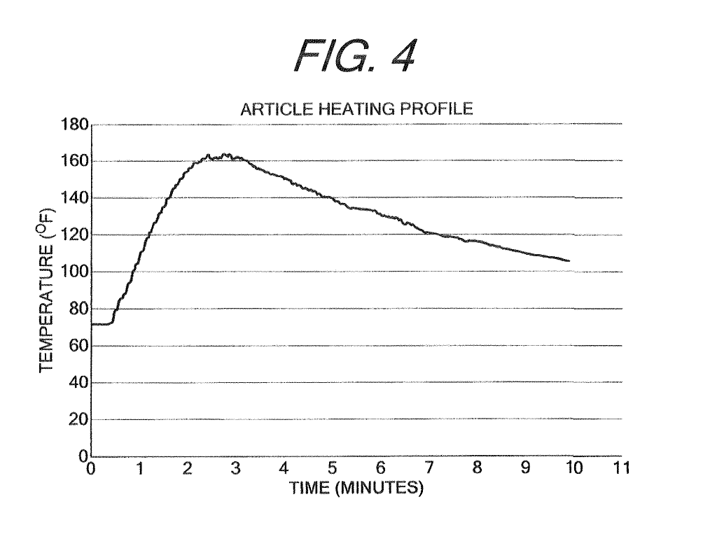

FIG. 4 shows the heating profile generated from a thermocouple attached to the center of an article described in FIG. 1. The heater mix in this example is composed of 80.6% Zn, 8.6% Carbon, 9.8% PTFE, and 1.0% H₂O. The heater mix was dosed with 30% amount by heater weight of 25% sodium bromide solution as the electrolyte. In this case the article is fabricated from an amorphous polylactic acid polymer (15% pattern void volume filled up with the heater mix) which softens above its glass transition temperature of approximately 140° F. The dry heater mix weight was 30 g with and the article weight being 45 g. It can be seen that the maximum temperature of the article and its embedded heater exceeds the softening temperature of the polymer within two minutes of heater activation, and quickly cools below the transition temperature by approximately five minutes in order to allow the thermoformable material to set.

While in the foregoing there has been set forth various embodiments of the invention, it is to be understood that the present invention may be embodied in other specific forms without departing from the spirit or central characteristics thereof. The present embodiments, therefore, are to be considered in all respects as illustrative and not restrictive, and the invention is not to be limited to the details given herein. While specific embodiments have been illustrated and described, numerous modifications come to mind without significantly departing from the characteristics of the invention and the scope of protection is only limited by the scope of the accompanying claims.

What is claimed is:

1. An article capable of being heated, the article comprising:
a material forming the article, the material having at least one cavity in a surface of the material;
at least one heater, the heater being embedded in the at least one cavity in the surface of the material; and
a sheet of porous material, the sheet of porous material being operatively engaged with the material so that the at least one cavity is covered and the at least one heater is held therein.

2. The article of claim 1 wherein the material forming the article is a thermoformable material and heat generated by the heater makes the thermoformable material moldable.

3. The article of claim 2 wherein the heater is a heater mix, the heater mix being an oxygen activated heater, and at least a portion of the heater mix is embedded in the at least one cavity.

4. The article of claim 3 further comprising an electrolyte, the electrolyte being included with the heater mix.

5. The article of claim 4 wherein the electrolyte comprises one or more from the group comprising sodium chloride, sodium bromide, potassium chloride, potassium bromide, or potassium hydroxide.

6. The article of claim 4 further comprising an airtight container, the airtight container being sealed around the article to prevent oxygen from contacting the material.

7. The article of claim 4 further comprising an insulator material, the insulator material at least partially surrounding and insulating the thermoformable material.

8. The article of claim 2 wherein the material is one or more from the group comprising poly(lactic acid), ethylene vinyl acetate, poly(caprolactone), poly(hydroxybutyrate), polyethylene, polypropylene, and polystyrene.

9. The article of claim 8 wherein the material contains an additive to improve the thermal conductivity of the material.

10. The article of claim 1 wherein the material is one or more from the group comprising woven and non-woven material.

11. The article of claim 1 wherein the heater can be activated using microwaves.

12. The article of claim 1 wherein the heater can be activated using an induction heating process.

13. The article of claim 3 wherein the heater mix includes zinc, carbon, and PTFE.

14. The article of claim 13 wherein the heater mix includes electrolyte.

15. The article of claim 14 wherein the electrolyte comprises one or more from the group comprising sodium chloride, sodium bromide, potassium chloride, potassium bromide, and potassium hydroxide.

16. The article of claim 13 wherein the heater mix includes a binder.

17. The article of claim 16 wherein the binder is one or more from the group comprising polytetrafluoroethyelene and polyethylene.

18. The article of claim 13 wherein the heater mix includes a filler.

19. The article of claim 18 wherein the filler is one or more from the group comprising sawdust, wood pulp, paper products, cotton linters, ground seed or nut hulls or products, plant cellular material, expanded perlite, vermiculite, diatomaceous earth, open-cell polyurethane foam, poly(acrylic acid), and hollow beads or spheres.

20. The article of claim 13 wherein the heater mix includes no more than 2% water.

21. The article of claim 13 wherein the heater mix has a bulk density in the range of 0.5 g/cm³ to 2.0 g/cm³.

22. The article of claim 1 wherein a plurality of cavities are formed in the material

23. The article of claim 22 wherein the plurality of cavities are configured in a honeycomb configuration.

24. The article of claim 23 wherein a portion of the heater is embedded in each of the plurality of cavities formed in the material.

25. The article of claim 24 wherein the combination of the material, cavities, porous sheet of material, and heater are arranged so that the article may be trimmed along a narrow access and a substantial portion of the heater may be retained within the plurality of cavities.

26. The article of claim 24 wherein a greater amount of heater is embedded in cavities located along a perimeter portion or edge of the material than the amount of heater embedded in the cavities in a middle portion or area of the material.

27. The article of claim 24 further comprising an electrolyte, wherein the electrolyte is added to the heater.

28. The article of claim 27 the amount or concentration of electrolyte added to any heater cavities located along a perimeter edge or portion of the material is greater than the amount or concentration of electrolyte added to any heater located in the middle portion or area of the material.

29. The article of claim 1 wherein the material is formed into one of a splint, an orthotic, a sleeve, a sock, a glove, a shirt, a vest, or a pant.

30. The article of claim 2 wherein the heater heats the thermoformable material to a moldable state in approximately 90 seconds.

31. The article of claim 30 wherein the temperature and heater drop below the temperature required to mold the thermoformable material in less than 10 minutes.

32. The article of claim 1 wherein the article retains a substantial portion of the heater is within the at least one cavity if the article is trimmed along a narrow axis.
